(19) United States
(12) Patent Application Publication
(10) Pub. No.: US 2002/0162125 A1
(43) Pub. Date: Oct. 31, 2002

(54) Methods and compositions for the modulation of neurogenic inflammatory pain and physical opiate withdrawal

(76) Inventors: Anne-Marie Salmon, Paris (FR); Susumu Sekine, Kanagawa (JP); Marina Picciotto, Guilford, CT (US); Jean-Pierre Changeux, Paris (FR)

Correspondence Address: Finnegan Henderson Farabow Garrett & Dunner, Suite 700, 1300 I Street, N.W., Washington, DC, 20005, US

(21) Appl. No.: 10/091,127

(22) Filed: Mar. 6, 2002

Related U.S. Application Data
(60) Provisional application No. 60/273,349, filed on Mar. 6, 2001.

(51) Int. Cl. A01K 67/027

(52) U.S. Cl. 800/3; 800/18

(57) ABSTRACT

A method of screening for a compound that is an antagonist of calcitonin gene related peptide (αCGRP) is provided. The method comprises: exposing a mutant mouse to a compound. The mutant mouse has a genome that comprises a homozygous disruption of the αCGRP gene, wherein the disruption results in the mutant mouse lacking detectable levels of endogenous αCGRP as compared to a wild type mouse. The response of the mutant mouse to a nociceptive-inducing stimulus is determined. A difference in response compared to a wild type mouse is indicative of the compound functioning to alter αCGRP activity. In a preferred embodiment, the disruption comprises the insertion of a transgene. A compound identified by the method is also provided. The compound is useful for ameliorating neurogenic inflammatory pain and/or physical opiate withdrawal.

### CROSS-REFERENCE TO RELATED APPLICATIONS

This application is based on and claims the benefit of U.S. Provisional Application No. 60/273,349, filed Mar. 6, 2001 (attorney docket no. 03495.6062) The entire disclosure of this application is relied upon and incorporated by reference herein.

### BACKGROUND OF THE INVENTION

The present invention relates to methods and compositions for the modulation of neurogenic inflammatory pain and/or physical opiate withdrawal. In a particular embodiment, the methods and compositions of the invention include methods and compositions for the specific inhibition of calcitonin gene related peptide (αCGRP).

Calcitonin gene related peptide (αCGRP) is expressed in a variety of cell types in both central and peripheral nervous systems and the characteristics of the gene encoding αCGRP have been disclosed (1). Among its various functions, αCGRP has been suggested to contribute to local, neurogenic inflammatory responses (2) and to nociception (3). αCGRP is expressed in 40% of the sensory neurons of the dorsal ganglia, being present in both the peripheral Aδ and C fibres and in the primary afferent nerves to the spinal cord.

Noxious thermal or mechanical stimulation evokes release of αCGRP in the superficial dorsal horn (4). In turn, αCGRP potentiates the local effects of other pain mediators, including substance P (3). Similarly injection of αCGRP in peripheral tissue can elicit visceral pain (5). In contrast, central administration of αCGRP in periacqueductal area and nucleus raphe magnus produces antinociceptive effects (6).

The putative role of αCGRP in nociception is further complicated by its relationship with opioids. Intrathecal αCGRP is known to decrease the analgesia produced by opioid agonists (7), whereas naloxone blocks the antinociceptive effects of αCGRP (6). Experiments with the αCGRP antagonist 8-37CGRP suggest that αCGRP contributes to the development of tolerance to the antinociceptive effect of morphine (8). Finally, αCGRP positive fibers are present in telencephalic areas involved in motivation, for instance the shell of nucleus accumbens and the central nucleus of the amygdala (9).

In summary, a great need exists for the definitive identification of compounds for the treatment of neurogenic inflammatory pain and/or physical opiate withdrawal.

### SUMMARY OF THE INVENTION

This invention aids in fulfilling these needs in the art.

The present invention relates, first, to methods and compositions for the modulation of neurogenic inflammatory pain and/or physical opiate withdrawal.

The compositions of the invention include, in one embodiment, compositions, including pharmaceutical compositions, for the specific inhibition of αCGRP activity. Such compositions of the present invention can include, but are not limited to, inhibitors of αCGRP gene activity, such as, for example, αCGRP antisense, triple helix and/or ribozyme molecules, and inhibitors of αCGRP activity.

In one embodiment, this invention provides a method of screening for a compound that is an antagonist of calcitonin gene related peptide (αCGRP). The method comprises: exposing a mutant mouse to a compound. The mutant mouse has a genome that comprises a homozygous disruption of the αCGRP gene, wherein the disruption results in the mutant mouse lacking detectable levels of endogenous αCGRP as compared to a wild type mouse. The response of the mutant mouse to a nociceptive-inducing stimulus is determined. A difference in response compared to a wild type mouse is indicative of the compound functioning to alter αCGRP activity. In a preferred embodiment, the disruption comprises the insertion of a transgene.

This invention also provides a compound, which is an antagonist of αCGRP, identified by the method of the invention.

Further, this invention provides a method for ameliorating neurogenic inflammatory pain comprising: administering a compound capable of specifically inhibiting αCGRP activity to an animal having neurogenic inflammatory pain symptoms in an amount sufficient to inhibit the αCGRP activity in the animal so that symptoms of neurogenic inflammatory pain are ameliorated.

Another method of the invention involves modulating physical opiate withdrawal comprising: administering a compound capable of specifically inhibiting αCGRP activity to an animal having physical opiate withdrawal symptoms for a time and in an amount sufficient to inhibit the αCGRP activity in the animal so that symptoms of physical opiate withdrawal are ameliorated.

Another method for modulating neurogenic inflammatory pain comprises: administering a compound capable of specifically inhibiting expression of αCGRP to an animal having neurogenic inflammatory pain symptoms for a time and in an amount sufficient to inhibit the expression of αCGRP in the animal so that symptoms of neurogenic inflammatory pain are ameliorated.

Another method of the invention involves modulating physical opiate withdrawal comprising: administering a compound capable of specifically inhibiting expression of αCGRP to an animal having physical opiate withdrawal symptoms for a time and in an amount sufficient to inhibit the expression of αCGRP in the animal so that symptoms of physical opiate withdrawal are ameliorated.

### BRIEF DESCRIPTION OF THE DRAWINGS

This invention will be described with reference to the drawings in which:

FIG. 1(A) shows the effects of capsaicin (20 μg) injected into the dorsal skin of the right hindpaw of αCGRP −/− mice and αCGRP +/+ mice (n=6/group), over 15 minutes. Licking manifestations started immediately after injection in both experimental groups.

FIG. 1(B) shows the effects of subcutaneous injections of formalin (20 μl of 2% paraformaldehyde in PBS) (n=10/group). The cumulative time spent licking the hindpaw during 0-5 minutes (acute phase) and 5-20 minutes (tonic phase) following injection is shown.

FIG. 1.(C) shows the results of induction of local edema by 20 μl of carrageenan (2% in PBS) injected into the dorsal skin of the right hindpaw measured as increased hindpaw thickness at 6 h (early time) and 72 h (delayed time) after injection (n=6/group). All data were analysed using Student's T-tests (* P<0.05; ** P<0.01 vs αCGRP +/+ mice).

FIG. 1.(D) shows the effects of acetic acid (10 μl/kg of 0.6% in water) or MgSO₄ (120 mg/kg) injected into the abdomen on writhing measured over 20 minutes post injection (n=10/group).

FIG. 2. shows the effects of (A) morphine (1 and 5 mg/kg, ip; n=10) and (B) nicotine (0.5 and 2 mg/kg, sc; n=8) in the tail flick assay, 30 min after drug injection. The results are presented as % MPE (maximum possible effect), where MPE=(test−control)/(cutoff−control)×100. Basal tail flick latencies were 1.73+/−0.13 in αCGRP +/+ mice and 1.79+/−0.12 in αCGRP −/− mice. Data were analysed using ANOVA followed by Tukey test (* P<0.05; ** P<0.01 vs αCGRP +/+ mice).

FIG. 3.(A) shows tolerance to morphine in the tail-flick assay: day 1 and 2, 50 mg/kg, day 3 and 4, 100 mg/kg; αCGRP −/− and +/+ mice did not differ in their development of tolerance to morphine analgesia. Acute morphine analgesia remained at the starting levels, respectively in −/− and +/+ mice treated with saline (not shown).

FIG. 3.(B) shows the results of heroin self-administration in αCGRP −/− (n=5) and αCGRP +/+ (n=5) mice (mean +/− SEM injections per session at each dose under a Fixed-Ratio 2 Time-Out 20 sec schedule of reinforcement, injection volume 50 μl over 2 sec). Following acquisition of stable heroin self-administration at 15.0 μg/kg/injection dose (3 consecutive sessions <+/−20% variation, >70% active lever responding), mice were allowed to self-administer heroin at each dose during two daily two-hr sessions in a Latin square dose order. After all heroin doses had been tested, saline vehicle was substituted for heroin until responding stabilized for at least 2 consecutive sessions (data points above “saline”). Intake of 30 μg/kg/injection dose differed significantly from a 7.5 and 15.0 μg/kg/injection doses, *, P<0.05 (means comparisons after appropriate two-way analysis of variance on self-administration).

FIG. 3.(C) shows the results of analysis of somatic signs of withdrawal: morphine dependence was induced by repeated ip morphine injections for one week: day 1 and 2, 50 mg/kg, day 3 and 4, 100 mg/kg; days 5-7, 100 mg/kg twice/day; day 8, 100 mg/kg in the morning. Then, withdrawal signs were precipitated by injection of naloxone (0.1 n=4,0.2 n=5 and 2.0 n=8 mg/kg, ip, 2 h after final morphine injection). αCGRP −/− mice exhibited significantly fewer total withdrawal signs than αCGRP +/+ mice (*, P<0.05, ** P<0.01). Naloxone (2 mg/kg, ip) in saline-treated control mice (mutant and wild type) did not induce any withdrawal signs (data not shown).

FIG. 3.(D) also shows the difference between αCGRP −/− mice and αCGRP +/+ mice when the opiate dependence syndrome was precipitated by naloxone in mice repeatedly injected with morphine. FIG. 3(D) shows that the frequency of each somatic sign of morphine withdrawal measured was reduced in the αCGRP (−/−) mice (jumping is presented as an example in FIG. 3D).

### DETAILED DESCRIPTION OF THE INVENTION

The present invention relates to methods and compositions for the modulation of neurogenic inflammatory pain and/or physical opiate withdrawal in mammals, including humans. More particularly, to establish the role of αCGRP, the behavioral responses of αCGRP null-mutant mice to chemical pain stimuli, mainly inflammatory, and to opiates was examined.

As used herein, “modulation” of neurogenic inflammatory pain and/or physical opiate withdrawal in mammals means increase or decrease of neurogenic inflammatory pain and/or physical opiate withdrawal in said mammals compared to such a pain and/or withdrawal in mammals to which the methods/compositions of the invention have not been applied/administered.

As used herein, “ameliorating” neurogenic inflammatory pain and/or physical opiate withdrawal means reducing symptoms of neurogenic inflammatory pain and/or physical opiate withdrawal.

It was previously shown that homozygous αCGRP mutant (−/−) mice, obtained by targeted disruption of exon 5 of the CT/CGRP gene, displayed a reduced antinociceptive tail-flick response to morphine (10). αCGRP (−/−) mice, backcrossed on the C57B1/6 strain to the 7th generation, were indistinguishable from wildtype αCGRP (+/+) mice in several spontaneous behaviors, including escape response latencies to acute thermal pain stimuli using both the hot-plate (55° C.) and tail-flick tests (10). It was concluded that αCGRP is not involved in acute pain sensation.

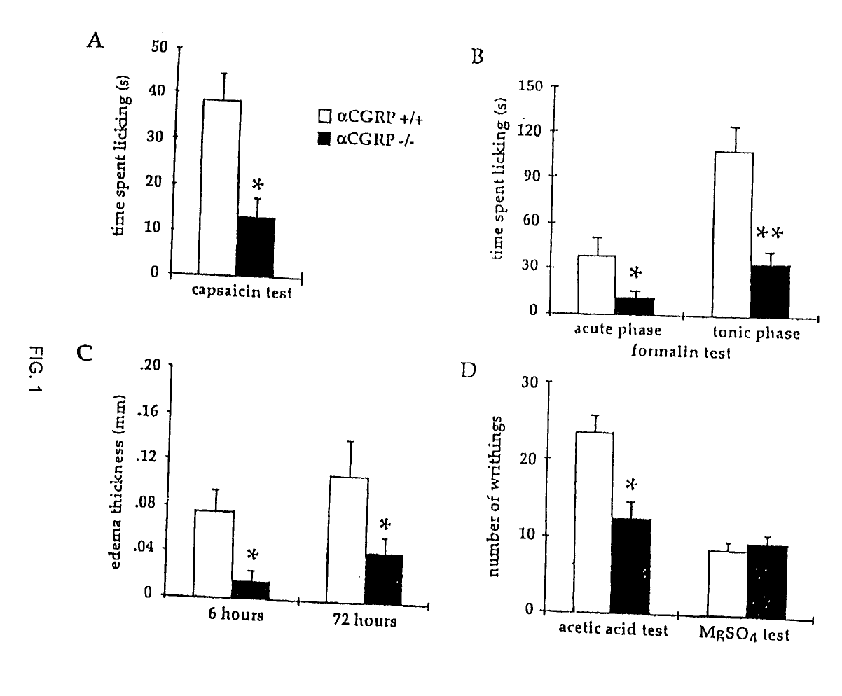

When chemical inflammatory pain was produced by capsaicin injection in the hindpaw, a procedure known to massively release αCGRP and substance P from nerve terminals, a significant attenuation of hindpaw licking response was found in αCGRP (−/−) mice (FIG. 1A). These mice also showed reduced hindpaw-licking time during the early (acute) phase of the formalin test, which provides a measure of direct chemical stimulation of the primary afferents. Hindpaw licking began immediately following capsaicin injections in both αCGRP −/− and αCGRP +/+ mice. Moreover a significant decrease in hindpaw-licking time was observed in αCGRP mutant mice during the second (tonic) phase of the formalin test (FIG. 1B). This second phase is thought to involve peripheral inflammatory events and ongoing tonic activation of nociceptors.

The contribution of the peripheral release of αCGRP to neurogenic inflammatory response was further confirmed by the profound reduction of edema produced by carrageenan injections in the hindpaw of αCGRP (−/−) mice when compared with αCGRP (+/+) (FIG. 1C).

Two models of visceral pain were also used: acetic acid that produces a delayed inflammatory response, and MgSO₄ that causes a non-inflammatory response. After intraperitoneal injections of acetic acid, significantly fewer episodes of writhing, a marker of intestinal discomfort, were observed in αCGRP (−/−) than in αCGRP (+/+) mice (FIG. 1D). In contrast, the episodes of writhing induced by intraperitoneal injections of MgSO₄ did not differ in the αCGRP (−/−) mice and αCGRP (+/+) mice.

Overall, these results indicate that αCGRP is critical for the production and, possibly, the transmission of somatic and visceral pain signals associated with neurogenic inflammation.

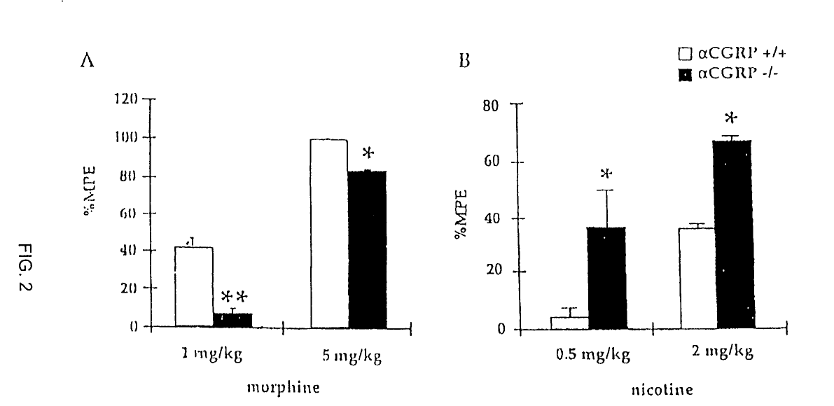

Next examined was the analgesic response to morphine and nicotine, two drugs known to act independently on nociception (11). In αCGRP −/− mice, the tail-flick responses were attenuated by 1 mg/kg and to a lesser extent by 5 mg/kg morphine (FIG. 2A), indicating that opiate effects on tail flick spinal reflex are only partially mediated by αCGRP. In contrast, the analgesic effects of nicotine were significantly increased at both 0.5 and 2.0 mg/kg dose in both tail-flick (FIG. 2B) and hot-plate test (data not shown), suggesting a possible decrease in αCGRP induced nicotinic acetylcholine receptor desensitization. These experiments also indicate that the descending pain inhibitory system of αCGRP (−/−) mice is not defective, but subject to graded activation.

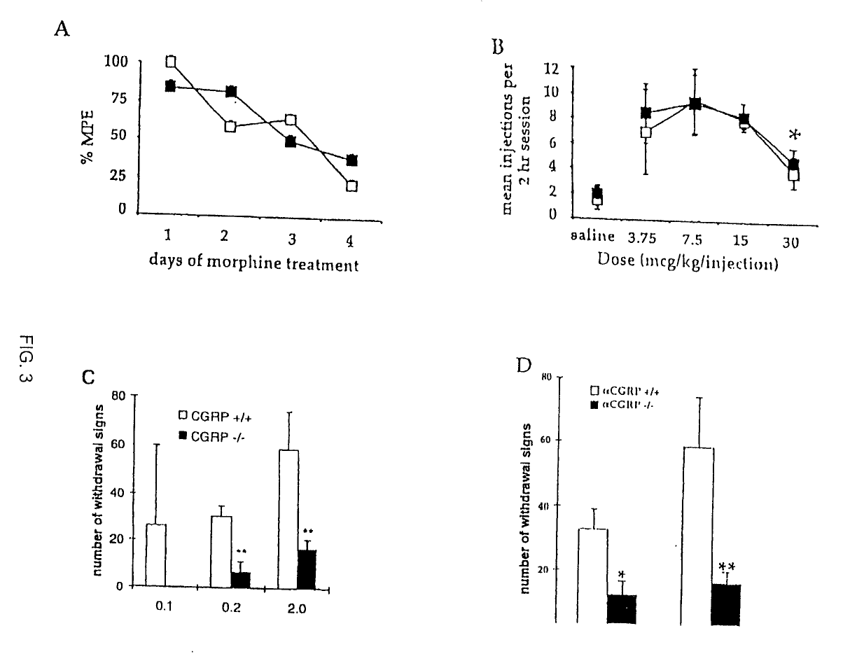

Tolerance to the antinociceptive effect of opiates was studied using a protocol of repeated morphine injections. The progressive decrease in the amplitude of morphine-elicited antinociceptive response in the tail-flick test did not differ between αCGRP (−/−) mice and αCGRP (+/+) mice (FIG. 3A), indicating that αCGRP is not involved in morphine tolerance.

To investigate whether the reinforcing properties of opiates were affected by the αCGRP mutation, αCGRP (−/−) and αCGRP (+/+) mice were trained to self-administer heroin in a discriminated lever press operant task. As shown in FIG. 3B, αCGRP (−/−) mice and αCGRP (+/+) mice did not differ in acquisition or maintenance of heroin self-administration, showing overlapping dose response curves, nor did they differ in acquisition of a food-reinforced lever press operant (data not shown). These data indicate that αCGRP does not contribute significantly to heroin reinforcement.

However, when the opiate dependence syndrome was precipitated by naloxone (0.1, 0.2 and 2.0 mg/kg) in mice repeatedly injected with morphine, a major difference between αCGRP (−/−) mice and αCGRP mice was observed (FIGS. 3C and D). Total withdrawal scores were significantly lower in αCGRP −/− mice across a range of naloxone dose (FIG. 3C). The frequency of each somatic sign of morphine withdrawal measured was also reduced in the αCGRP −/− mice (jumping is presented as an example in FIG. 3D). The opiate withdrawal syndrome is known to produce a general malaise and intense aversive emotional state (12). It is hypothesized to be generated by adaptive mechanisms to the prolonged exposure to exogenous opiates. Recent evidence indicates that attenuation of both neurogenic inflammatory responses and physical opiate withdrawal syndrome is also observed in mice with targeted deletion of the substance P NK1 receptor (13).

The present results indicate that an important component of the withdrawal malaise is the peripheral nervous system that mediates neurogenic inflammatory responses. This system can be modulated by increased autonomic output observed during opiate withdrawal resulting in amplification of peripheral neurogenic pain signals. In turn, increased pain signals from the periphery can augment the withdrawal malaise by enhancing both the affective and somatic components of opiate withdrawal. The contribution of peripheral signals to emotional processing is a well established phenomenon (14). This phenomenon can extend to the interpretation of the opiate dependence syndrome, initially supported by data from targeted deletion of both NK1 and CT/CGRP genes in mice. Independently from this view, the present results indicate that αCGRP antagonists can serve as a treatment of both neurogenic inflammatory pain and physical opiate withdrawal.

Described herein are compounds, including pharmaceutical compositions, which can be utilized for the amelioration of neurogenic inflammatory pain and/or physical opiate withdrawal. More specifically, said compounds are antagonists of calcitonin gene related peptide (αCGRP). Such compounds can include, but are not limited to, small peptides, small organic molecules, antisense, and triple helix molecules. Compositions can include polyclonal and/or monoclonal antibodies for the modulation of such pain and/or withdrawal symptoms.

A variety of methods can be utilized for the identification of the compounds of the invention. The identification methods comprise isolated protein-based assays, cell-based assays, and whole animal assays.

Assays that serve to test inhibition capacity in an in vivo situation are preferred. Typically, such assays include administering to an animal a test compound and measuring its effect. With respect to inhibitors of neurogenic inflammatory pain and/or physical opiate withdrawal, animal assays using rodents are preferred. As described herein, a variety of assays can be employed, including the hot-plate assay, the tail-flicks assay, the hindpaw capsaisin injection assay, the carrageenan rat paw edema assay, the acetic acid assay for visceral pain, and/or the MgSO₄ assay for visceral pain. Literature citations describing how these assays are carried out are as follows:

“Calcitonin Gene Related Peptide (αCGRP) in capsaicin-Sensitive Substance P-Immunoreactive Sensory Neurons in Animals and Man: Distribution and Release by Capsaicin.”

Franco-Cereda A., Henke H., Lundberg J. M., Peterman J. B., Hökfelt T., and Fischer J. A., Peptides, 8, 399-410, 1987.

“Differential contribution of the two phases of the formalin test to the pattern of C-fos expression in the rat spinal cord: studies with remifentanil and lidocaine.” Abbadie C., Taylor B. K., Peterson M. A., and Basbaum A. I., Pain, 69, 101-110, 1997.

“A method for determining loss of pain sensation.” D'Amour F. E., and Smith D. L., J. Pharmacol. Exp. Ther., 72, 74-79, 1941.

“Induction of cyclooxygenase-2 causes an enhancement of writhing response in mice.” Matsumo H., Naraba H., Ueno A., Fujiyoshi T., Murakami M., Kudo I., and Oh-ishi S., Eur. J. Pharmacol., 352 (1), 47-52,1998.

Compounds that ameliorate neurogenic inflammatory pain and/or physical opiate withdrawal can also be tested in cell-based assays to test inhibitory capacity within the cell. Such cell-based assays can be utilized to test a number of features of a potential inhibitor, including, for example, the compound's ability to enter the cell, its cytotoxicity, as well as its ability to act as an inhibitor once inside the cell. Further, cell-based assays can function to identify compounds that act more indirectly to inhibit αCGRP activity.

A typical cell-based assay can involve contacting a cell expressing the activity of interest with a test compound for a time and measuring the inhibition of such an activity. For measurements, for example, whole cells can be lysed according to standard techniques and tested for the presence of αCGRP activity.

Among the inhibitors of the invention are nucleic acid antisense and/or triple helix molecules that act to inhibit expression of the αCGRP gene involved in one or more of the activities relating to neurogenic inflammatory pain and/or physical opiate withdrawal processes. Such inhibitors can be utilized in methods for the amelioration of neurogenic inflammatory pain and/or physical opiate withdrawal.

Antisense approaches can be utilized to inhibit or prevent translation of mRNA transcripts; triple helix approaches to inhibit transcription of the gene of interest itself. Antisense approaches involve the design of oligonucleotides (either DNA or RNA) that are complementary to αCGRP mRNA. The antisense oligonucleotides bind to the complementary mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex. In the case of double-stranded antisense nucleic acids, a single-strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex. Antisense nucleic acids should be at least six nucleotides in length, and are preferably oligonucleotides ranging from 6 to about 50 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups, such as peptides (e.g., for targeting host cell receptors in vivo), or agents facilitating transport across the cell membrane. To this end, the oligonucleotide may be conjugated to another molecule, e.g., a peptide, hybridization triggered cross-linking agent, transport agent, hybridization-triggered cleavage agent, etc. Oligonucleotides of the invention can be synthesized by standard methods known in the art, e.g. by use of an automated DNA synthesizer (such as are commercially available from Biosearch, Applied Biosystems, etc.).

Any of the compounds identified via the techniques described herein can be formulated into pharmaceutical compositions and utilized as part of the amelioration methods of the invention. Such pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or topical, oral, buccal, parenteral or rectal administration.

For oral administration, the pharmaceutical compositions can take the form of, for example, tablets or capsules prepared by conventional means with pharmaceutically acceptable excipients, such as binding agents (e.g., pregelatinised maize starch, polyvinylpyrrolidone or hydroxypropyl methylcellulose), fillers (e.g., lactose, microcrystalline cellulose or calcium, hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base, such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions, or emulsions in oily or aqueous vehicles, and can contain formulatory agents, such as suspending, stabilizing, and/or dispersing agents. Alternatively, the active ingredient can be in a powder form for constitution with a suitable vehicle, e.g., sterile pyrogen-free water, before use.

The compounds can also be formulated in rectal compositions, such as suppositories or retention enemas, e.g., containing conventional suppository bases, such as cocoa butter or other glycerides.

All preparations can be suitably formulated to give controlled release of the active compound. For example, in addition to the formulations described previously, the compounds can also be formulated as a depot preparation. Such long acting formulations can be administered by implantation (for example subcutaneously or intramuscularly) or by intramuscular injection. Thus, for example, the inhibitors can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device, which may contain one or more unit dosage forms containing the active ingredient. The pack can, for example, comprise metal or plastic foil, such as a blister pack. The pack or dispenser device can be accompanied by instructions for administration.

In general, the inhibitors and compositions of the invention can be utilized for modulating a neurogenic inflammatory pain and/or physical opiate withdrawal. A method for ameliorating symptoms of a neurogenic inflammatory pain and/or physical opiate withdrawal, can, for example, comprise: contacting a cell with an αCGRP inhibitor for a time and in an amount sufficient to inhibit αCGRP activity so that symptoms of expression of αCGRP the neurogenic inflammatory pain and/or physical opiate withdrawal are ameliorated. The αCGRP inhibitor can include, but is not limited to, the αCGRP inhibitor compositions, including pharmaceutical compositions.

The compounds and compositions to be administered as part of methods for ameliorating symptoms of neurogenic inflammatory pain and/or physical opiate withdrawal according to the invention are administered to a patient at therapeutically effective doses to treat or ameliorate symptoms of such disorders. A therapeutically effective dose refers to that amount of the compound sufficient to result in amelioration of symptoms of neurogenic inflammatory pain and/or physical opiate withdrawal.

Toxicity and therapeutic efficacy of the inhibitors can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

Data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any inhibitor used in the method of the invention, the therapeutically effective dose can be estimated initially from rodent assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans.

This invention will be more fully understood by reference to the following Example.

### EXAMPLE

The following methods were employed in the experiments reported herein.

Development of αCGRP −/− and αCGRP +/+ Mice.

The targeting construct was performed from the CT/CGRP gene (15), and the chimera mice were obtained as described (15). αCGRP −-− and +/+ mice are derived from backcrosses on the C57B16 strain after mating of heterozygous +/− mice. Homozygous mutant mice, from all generations, are healthy, fertile and do not present obvious abnormalities. The body temperature is the same in mutant and wild type mice, and no differences in the body weight of the two lines were observed during development.

Behavioral Studies.

Thermal Stimuli.

The tail flick assay was performed with a tail flick analgesia meter from Columbus Instruments. The cut-off time of the test was 10 seconds. In the hot plate assay, the mice were analyzed with a Basile Ugo apparatus; the latency to forepaw licking or jumping at 55° C. was noted. The cut-off time of the experiment was 30 seconds.

Chemical Stimuli.

20 μl of capsaicin was injected in the right hindpaw and the time spent licking within 15 minutes after injection was noted. For the formalin test, the inventors injected 20 μl of dilute formalin (2% of paraformaldehyde in PBS) under the dorsal skin of the right hindpaw and measured the licking time during two periods, 0 to 5 minutes and 5 to 20 minutes, post injection. Visceral pain responses were analyzed after acetic acid (10 ml/kg of 0.6% dilution) of MgSO₄ (120 mg/kg), ip injection; the number of writings within 20 minutes post injection were counted.

Inflammatory Test.

20 μl of carrageenan solution (2% in PBS) was injected under the dorsal skin of the right hindpaw. The thickness of the edema was estimated as the difference in the thickness of the right hindpaw before and after injection.

Drug Analgesia.

Morphine or nicotine analgesia were analyzed by using tail flick and hot plate tests. The tests were performed 30 minutes after the injection of drug on αCGRP −/− and +/+ mice. Low and high concentrations of drugs were tested: 1 mg and 5 mg/kg for morphine in ip, and 0.5 mg and 2 mg/kg for nicotine in sc.

Morphine Tolerance.

Daily injections ip of morphine were performed: day 1 and 2, 50 mg/kg, day 3-4, 100 mg/kg. Analgesia morphine was tested. αCGRP −/− and +/+ mice were analyzed in parallel.

Heroin Self-Administration.

All procedures involving animals described here were approved by the Service Vétérinaire of the Canton of Vaud, Lausanne, Switzerland. Six individually housed mice of each genotype (CGRP −/− and +/+) were tested during the light phase (12/12; lights on 7:00 am) in daily sessions 5 days/week. Operant chambers housed in sound-attenuating cubicles with a house light and ventilation fan were equipped with cue lights above two retractable levers, a liquid dipper, syringe pump, liquid swivel and counterbalance arm (swivel and counterbalance arms—Instech, Plymouth Meeting, Pa., USA; all other equipment—MedAssociates, Georgia, Vt., USA). A single lever press on the “active” lever raised the dipper into the cage for 10 s and illuminated the cue light above the lever during reinforcer presentation and for a 1-10 s post-reinforcer Time-out (TO) period, depending on the schedule of reinforcement. Lever presses on the “active” lever (counterbalanced between subjects) while the dipper was raised or during the TO as well as “inactive” lever presses at any time were counted but had no experimental consequences. Mice deprived of food for 16 hours were first trained to press the “active” lever for sweetened milk reinforcers (17 μl, 3.8% fat Pasteurized milk with 60 g/L sucrose) under a fixed-ratio 1 (FR1) schedule of reinforcement with a 1 s TO. The TO was increased between sessions to 5 s and then 10 s and finally the FR was increased to 2, up to a final schedule of FR2 Time-out 10 s, after mice had successfully earned 50 milk reinforcers in single, daily 1-hr. sessions under each schedule of reinforcement. Mice were given 3 g of food in their home cages after each session until the end of food training when they were returned to food ad libitum. After completion of food training, mice were implanted with chronic indwelling jugular catheters. Catheter construction was as described previously with minor modifications. Daily 2-hr. heroin self-administration sessions began at least 48 hours after surgery under an FR2 schedule of reinforcement with an 18 s post-injection TO period (15 μg/kg/injection, delivered in 50 μl over 2s). Following acquisition of stable heroin self-administration (+20% variation in the total number of heroin injections earned per session for 3 consecutive sessions and minimum 70% active vs. inactive lever responding), mice were allowed to self-administer each different dose of heroin (3.75, 7.5, 15.0 or 30.0 μg/kg/injection) for 2 consecutive sessions in a within subjects Latin square design. After each mouse had been tested with all doses, saline vehicle was substituted for heroin in daily 2-hr. sessions until responding extinguished (3-5 sessions).

Morphine Dependence.

Morphine dependence was induced by repeated ip morphine injection for one week: day 1 and 2, 50 mg/kg; day 3 and 4, 100 mg/kg; days 5-7, 100 mg/kg twice/day; day 8, 100 mg/kg in the morning. Then, withdrawal signs were precipitated by injection of naloxone (2 mg/kg, ip, 2 h after final morphine injection). The inventors noted the behavioral withdrawal symptoms (jumping, teeth chattering, tremors, paw tremors, wet dog shakes, ptosis, diarrhea) during 30 minutes after naloxone injection.

A plasmid containing a targeted deletion of exon 5 of the CT/CGRP gene using CMV-LacZ genes has been deposited at the Collection Nationale de Cultures de Microorganismes (“C.N.C.M.”) 28, rue du Docteur Roux, 75724 Paris Cedex 15, France as follows:

| Plasmid | Accession No. | Deposit Date |
|---|---|---|
| E.C.XL1 Blue; | I-2365 | Dec. 8, 1999. |
| CMV5'At3'/[alpha] | | |
| 5 CGRP | | |

This plasmid is useful as a targeting construct for the production of αCGRP −/− mice for use in the invention.

In summary, the neuropeptide αCGRP is involved in the complex process of pain signaling. Yet the precise contribution of αCGRP remains unclear. This invention shows that mice lacking αCGRP display an attenuated response to capsaicin, formalin, carrageenean, and acetic acid-induced neurogenic inflammatory pain. αCGRP mutant mice showed attenuated analgesic effects of morphine at low doses, no change in tolerance to morphine antinociceptive properties, and no shift in heroin self-administration dose-response curve. In contrast, they display a marked decrease of physical opiate withdrawal syndrome precipitated by naloxone. Taken together these results show that αCGRP plays a critical role in mediating both neurogenic inflammatory responses and sensitivity to morphine withdrawal, supporting the potential key role of neurogenic pain substrate in determining the severity of opiate withdrawal syndrome.

### REFERENCES

The following publications have been cited herein. The entire disclosure of each publication is relied upon and incorporated by reference herein.

1. M. G. Rosenfeld, S. G. Amara and R. M. Evans,., Science, 225,1315-1320,1984.

2. S. R. Hughes and S. D. Brain, Br. J. Pharmacol., 104, 738-742,1991.

3. I. L. Gibbins, J. B. Furness. and M. Costa, Cell Tissue Res., 248, 417-437 1987.

4. C. R Morton, and W. D. Hutchison, Neuroscience, 31, 807-815,1989.

5. Friese, L. Diop, E. Chevalier, F. Angel, P. J. River and S. G. Dahl, Regul. Pept., 70, 1-7,1997.

6. Y-H. Huang, G. Brodda-Jansen, T. Lundeberg and L-C. Yu, Brain Res., 873, 54-59, 2000.

7. P. Welch, A. K. Singha, and W. L. Dewey, J. Pharmacol. Exp. Ther., 251, 1-8, 1989.

8. D. P. Menard, D. van Rossum, S. Kar, and R. Quirion, Can. J. Physiol. Pharmacol., 73, 1089-1095,1995.

9. T. Hökfelt, U. Arvidsson, S. Ceccatelli, R. Cortés, S. Cullheim, Å. Dagerlind, H. Johnson, C. Orazzo, F. Piehl, V. Pieribone, M. Schalling, L Terenius, B. Ulfhake, V. M. Verge, M. Villar, Z. Wiesenfeld-Hallin, X-J. Xu, and Z. Xu, Ann. NY Acad. Sci., 657,119-134,1992.

10. A. M. Salmon, M. I. Damaj, S. Sekine, M. R. Picciotto, L. M. Marubio and J. P. Changeux, NeuroReport, 10, 849-854, 1999.

11. L. M. Marubio, M. Del Mar Arroyo-Jimenez, M. Cordero-Erausquin, C. Léna, N. Le Novére, A. De Kerchove d'Exaerde, M. Huchet, M. I. Damaj and J. P. Changeux, Nature, 398, 805-810,1999.

12. G. Schulteis, A. Markou, L. H. Gold, L. Stinus, and G. F. Koob, J. Pharmacol. Exp. Ther., .271,1391-1398,1994.

13. P. Murtra, A. M. Sheasby, S. P. Hunt and C. De Felipe, Nature, 405,180-183,2000.

14. A. R. Damasio, Descartes' Error: Emotion, Reason, and the Human Brain, (New-York; Avon Books, 1995).

What is claimed is:

1. A method of screening for a compound that is an antagonist of calcitonin gene related peptide (αCGRP), said method comprising:
(A) exposing to said compound a mutant mouse, whose genome comprises a homozygous disruption of the αCGRP gene, wherein said disruption results in said mutant mouse lacking detectable levels of endogenous αCGRP as compared to a wild type mouse; and
(B) determining the response of said mutant mouse to a nociceptive-inducing stimulus, wherein a difference in response compared to a wild type mouse is indicative of the compound functioning to alter αCGRP activity.

2. The method of claim 1, wherein the disruption of (A) comprises the insertion of a transgene.

3. The method as claimed in claim 1, wherein said method comprises determining said response by the tail-flick method and said compound inhibits αCGRP activity in the mutant mouse.

4. The method as claimed in claim 1, wherein said method comprises determining said response by the hot plate method and said compound inhibits αCGRP activity in the mutant mouse.

5. The method of claim 1, wherein said method comprises determining said response by carragenan rat paw edema assay and said compound inhibits αCGRP activity in the mutant mouse.

6. A compound, which is an antagonist of αCGRP, identified by the method of any one of claims 1 to 5.

7. The compound of claim 6, which is a peptide, small organic molecule, antisense molecule, or a triple helix molecule.

8. The compound of claim 6, which is a monoclonal antibody.

9. A method for ameliorating neurogenic inflammatory pain comprising:
administering a compound capable of specifically inhibiting αCGRP activity to an animal having neurogenic inflammatory pain symptoms in an amount sufficient to inhibit the αCGRP activity in the animal so that symptoms of neurogenic inflammatory pain are ameliorated.

10. A method for ameliorating physical opiate withdrawal comprising:
administering a compound capable of specifically inhibiting αCGRP activity to an animal having physical opiate withdrawal symptoms in an amount and for a time sufficient to inhibit the αCGRP activity in the animal so that symptoms of physical opiate withdrawal are ameliorated.

11. A method for ameliorating neurogenic inflammatory pain comprising:
administering a compound capable of specifically inhibiting expression of αCGRP to an animal having neurogenic inflammatory pain symptoms in an amount and for a time sufficient to inhibit the expression of αCGRP in the animal so that symptoms of neurogenic inflammatory pain are ameliorated.

12. A method for ameliorating physical opiate withdrawal comprising:
administering a compound capable of specifically inhibiting expression of αCGRP to an animal having physical opiate withdrawal symptoms in an amount and for a time sufficient to inhibit the expression of αCGRP in the animal so that symptoms of physical opiate withdrawal are ameliorated.

13. The compound of claim 6, wherein said compound specifically inhibits the αCGRP activity.

14. The compound of claim 6, wherein said compound specifically inhibits the αCGRP expression.

15. A pharmaceutical composition comprising the compound of claim 6 along with at least one physiologically acceptable carrier or excipient.

16. A compound for ameliorating neurogenic inflammatory pain an/or physical opiate withdrawal, wherein said compound is an antagonist of calcitonin gene related peptide αCGRP.
